(19) United States
(12) Patent Application Publication
(10) Pub. No.: US 2005/0049509 A1
(43) Pub. Date: Mar. 3, 2005

(54) Cervix monitoring system and related devices and methods

(76) Inventors: Hebah Noshy Mansour, La Mirada, CA (US); Ramez Emile Necola Shehada, La Mirada, CA (US)

Correspondence Address: CHRISTIE, PARKER & HALE, LLP, PO BOX 7068, PASADENA, CA, 91109-7068, US

(21) Appl. No.: 10/931,013

(22) Filed: Aug. 30, 2004

Related U.S. Application Data
(60) Provisional application No. 60/498,496, filed on Aug. 28, 2003.
(60) Provisional application No. 60/576,479, filed on Jun. 2, 2004.
(60) Provisional application No. 60/587,530, filed on Jul. 13, 2004.

(51) Int. Cl. A61B 5/00; A61B 5/103

(52) U.S. Cl. 600/476; 600/591

(57) ABSTRACT

The present invention is directed to a minimally invasive system and method for monitoring changes in a cervix opening during labor, including changes in its diameter. The system having a probe and a monitoring unit serve to monitor periodically the cervix opening during labor and obtain measurements of the diameter of the cervix opening. The probe primarily includes a camera for imaging the cervix opening, a lens to provide an optimal field of view for the camera at close range to the cervix opening, a light source to illuminate the cervix and a balloon to expand the vagina around the probe and position the probe to allow unobstructed imaging of the cervix. The monitoring unit which controls the probe to acquire images of the cervix and receives and processes image data from the probe includes a processor which uses image segmentation techniques to identify and measure the opening of the cervix and generates data signals. The monitoring unit also includes an image display which displays information based on the data signals for viewing by health care providers and/or the patient, including graphic images of the cervix opening and other text information relating to the cervix opening as an indicator of the progress of labor. In one embodiment, the processor implements segmentation techniques and/or blob analysis algorithms to analyze the images of the cervix to identify the opening and measure its diameter. Other algorithms may also be implemented to correct for any spatial distortion, particularly barrel distortion.

### CROSS REFERENCE TO RELATED APPLICATIONS

This application claims the benefit of U.S. Provisional Application No. 60/498,496, filed Aug. 28, 2003, entitled Method and Devices for Cervix Monitoring; U.S. Provisional Application No. 60/576,479, filed Jun. 2, 2004, entitled Ranging and Imaging Methods for Cervix Monitoring; and U.S. Provisional Application No. 60/587,530, filed Jul. 13, 2004, entitled Probes for Cervix Monitoring, the entire contents of which applications are incorporated herein by reference.

### FIELD OF THE INVENTION

The present invention relates to a method and system for monitoring progress of labor during childbirth, in particular, changes in the cervix, including changes in the size of the opening of the cervix, during childbirth.

### BACKGROUND OF THE INVENTION

Monitoring the progress of cervix dilation is desirable during labor because the progress of cervix dilation can be an indicator of various conditions or factors including: (1) arrested or dysfunctional labor, (2) cephalopelvic disproportion, (3) when and whether a Caesarean-section should be performed, (4) when and whether labor inducing/enhancing drugs (e.g. Pitocin) should be used, (5) when should different anesthetic or analgesic agents be administered, and/or (6) when the patient should begin pushing.

In current clinical practice, cervix dilation (or dilatation, used interchangeably herein) is typically, if not always, monitored manually by a health care provider who inserts a gloved hand into the patient's vagina and then uses his or her fingers to probe and assess the diameter of the cervix opening. This method is known as digital probing and suffers several inherent limitations, including the following. First, the method is approximate as the accuracy of the measurement depends on the experience of the health care provider. Second, the method can cause discomfort to the patient during each session of digital probing which may be performed repeatedly at an interval of 30 minutes or so throughout labor. Third, the health care provider may forget to perform the exam or even avoid it intentionally if the patient is sleeping. Fourth, hand examinations can increase the risk of infection to the patient and the fetus especially if the fetal membranes have been ruptured. In the latter regard, hand examinations may cause complications, including neonatal sepsis to the infant, chorioamnionitis to the fetal membranes, and/or endomyometritis to the uterine muscles.

As such, hand examinations are often minimized and hence do not provide continuous reliable monitoring of the progress of the labor. In many clinical cases, engagement of the fetus head in the cervix at station +3 (where the presenting part is in the perineum) go unnoticed, especially where the patient is under epidural anesthesia. All of the above factors make desirable the development of an automated technique to continuously, or at least intermittently, monitor cervix dilation and provide an improved, if not more accurate, measurement of the diameter of the cervix opening.

Current methods for measuring the diameter of the cervix opening during labor are reviewed by Lucidi et al. (Lucidi, R. Scott; Blumenfeld, Lee A.; Chez, Ronald A., “Cervimetry: A Review of Methods for Measuring Cervical Dilatation During Labor,” Obstetrical and Gynecological Survey, Volume 55(5), pp 312-320, May 2000) who wrote “although many instruments have been developed to measure cervical dilatation during labor and their use as a research tool has been established, no device has yet been successfully used for clinical obstetrics. The ideal device has not yet been developed; however, because repeated digital cervical examinations are time consuming for the clinician, are poorly reproducible, and are uncomfortable for the patient, continued efforts to develop a cervimeter suitable for clinical use is a worthwhile endeavor.”

As quoted above, there have been many attempts to develop devices for accurate and user-independent monitoring of the diameter of the cervix opening. However, previous techniques failed to gain clinical acceptance due to several limitations, including the complexity of installation, inaccuracy of measurements, tissue trauma caused by the devices or their components, including the manner by which the components are attached to the cervix, blockade of the cervical canal, costly sterilization between uses, and/or patient discomfort. Consequently, the manual method of digital probing continues to be a favored method of monitoring cervix dilation. Therefore, there exists a desire for a system and method which monitor the diameter of the cervix opening during labor in a noninvasive manner and preferably uses a probe that can remain in the vaginal region throughout the course of labor.

### SUMMARY OF THE INVENTION

The present invention is directed to a minimally invasive system and method for monitoring changes in a cervix opening during labor, including changes in its diameter. The system having a probe and a monitoring unit serve to monitor periodically the cervix opening during labor and obtain measurements of the diameter of the cervix opening. The probe primarily includes a camera for imaging the cervix opening, a lens to provide an optimal field of view for the camera at close range to the cervix opening, a light source to illuminate the cervix and a balloon to expand the vagina around the probe and position the probe to allow unobstructed imaging of the cervix. The balloon is triggered to inflate when images of the cervix opening are taken and can deflate when the camera is not capturing images. A tail or handle portion of the probe facilitates the insertion and removal of the probe from the vagina and protects electrical and pneumatic connections between the probe and the monitoring unit. The tail portion is generally flexible but is adapted to have sufficient rigidity by means of a rod applicator, bead trains or an inflatable body to facilitate the insertion of the probe into the vagina.

Advantageously, the probe in one embodiment is configured to provide a predetermined distance from which the camera has an appropriate range to image the cervix opening. In one embodiment, a housing of the probe is configured to generally abut the cervix such that the camera fixedly situated in the housing is positioned at the predetermined distance from the cervix.

In another embodiment, the probe has a disposable portion releasably coupled to a reusable portion. In a more detailed embodiment, a protective sheath extends from a distal disposable portion of the probe to cover and protect a proximal reusable portion.

The probe may also have a compartmental balloon or multiple balloons which are symmetrically or asymmetrically disposed around the probe and can inflate simultaneously or separately to selectively orient and position the probe in the vagina. The inflated balloons may also create drainage channels across the probe to allow fluids to flow from the cervix and out of the vagina.

The present invention also has a probe module that is supported on a distal end of an applicator which releasably holds a probe module until the module has been placed in the vagina at an appropriate distance from the cervix. To that end, the applicator has a range-adjusting structure which may be a coded scale ranging means, a swivel arm means, a string limiter means or an automatic range-adjusting means. In another embodiment, the module itself may carry an optical range means to determine the range between the module and the cervix, which also employs the camera to capture an image of a spot of illumination created by an optical beam of a known spread rate.

The monitoring unit which controls the probe to acquire images of the cervix and receives and processes image data from the probe includes a processor which uses image segmentation techniques to identify and measure the opening of the cervix and generates data signals. The monitoring unit also includes an image display which displays information based on the data signals for viewing by health care providers and/or the patient, including graphic images of the cervix opening and other text information relating to the cervix opening as an indicator of the progress of labor. In particular, the information display includes measured cervix diameter and dilation rate. One or more monitoring systems may be networked to a remote nursing station to allow the simultaneous monitoring of multiple patients.

In one embodiment, the processor implements segmentation techniques and/or blob analysis algorithms to analyze the images of the cervix to identify the opening and measure its diameter. Other algorithms may also be implemented to correct for any spatial distortion, particularly barrel distortion. In another embodiment, a portable controller is provided to facilitate the mobility of the patient, by eliminating a cable extending between the probe and the monitoring unit.

### BRIEF DESCRIPTION OF THE DRAWINGS

These and other features and advantages of the present invention will be better understood by reference to the following detailed description when considered in conjunction with the accompanying drawings wherein:

FIG. 1 is a schematic diagram showing an embodiment of a monitoring system having a self-ranging probe inside the vagina and connected to a monitoring unit.

FIG. 2A is a front view of the self-ranging probe of FIG. 2B.

FIG. 2B is a side view of the self-ranging probe of FIG. 1 with a deflated balloon.

FIG. 2C is a front view of the self-ranging probe of FIG. 2D.

FIG. 2D is a side view of the self-ranging probe of FIG. 1 with an inflated balloon.

FIG. 2E is a side view of a rod applicator of the self-ranging probe of FIG. 2B.

FIGS. 2F and 2G are partial side views of an embodiment of a probe handle with bead trains.

FIG. 2H is a partial side view of another embodiment of a probe handle with an inflatable support structure.

FIG. 2I is a cross-sectional view of the flexible tube of FIG. 2B taken along lines I to I.

FIG. 3A is a side view of an embodiment of a disposable cap for use with a reusable self-ranging probe.

FIG. 3B is a side view of an embodiment of a reusable core for use with the cap of FIG. 3A.

FIG. 3C is a side view of the cap and core of FIGS. 3A and 3B releasably coupled to each other.

FIG. 4A is a block diagram of an embodiment of the monitoring system of the present invention.

FIG. 4B shows a network of multiple monitoring systems communicating with a common nursing station.

FIG. 5A shows an embodiment of a graphic user interface and display screen of a monitoring unit.

FIG. 5B shows an alternative embodiment of a graphic user interface and display screen of the monitoring unit.

FIGS. 6A and 6B are photographic images of a normal female cervix with an opening and a normal female cervix with a larger opening.

FIGS. 6C and 6D are the photographic images of FIGS. 6A and 6B where the cervix openings have been automatically segmented, measured and outlined in accordance with the present invention.

FIG. 7 is a block diagram of an embodiment of a monitoring system of the present invention having a controller unit.

FIG. 8A is a front view of the inflatable-spacer probe of FIG. 8B.

FIG. 8B is a side view of an embodiment of an inflatable-spacer probe.

FIG. 8C is a front view of the inflatable-spacer probe of FIG. 8D.

FIG. 8D is a side view of an alternative embodiment of an inflatable-spacer probe with drainage members.

FIG. 9A is a front-view of the multi-balloon probe of FIG. 9B deflated.

FIG. 9B is a side-view of an embodiment of a deflated multi-balloon probe.

FIG. 9C is an end-view of the multi-balloon probe of FIG. 9B.

FIG. 9D is a front-view of the multi-balloon probe of FIG. 9B inflated.

FIG. 9E is a side-view of the multi-balloon probe of FIG. 9B inflated.

FIG. 9F is an end-view of the multi-balloon probe of FIG. 9B.

FIGS. 9G and 9H are front and side views of another embodiment of a multi-balloon probe with an air nozzle.

FIG. 10 is an isometric-view of the multi-balloon probe of FIG. 9B attached to an applicator.

FIG. 11A is a side-view of an embodiment of a coded scale range-adjusting applicator.

FIG. 11B is a side-view of an embodiment of a swivel arm range-adjusting applicator.

FIG. 11C is a side-view of an embodiment of a string limiter range-adjusting applicator.

FIG. 11D is a side-view of an embodiment of an automatic range-adjusting applicator.

FIG. 11E is a perspective view of the applicator of FIG. 11C.

FIG. 12A is a side-view of an embodiment of a probe with range finding capability while measuring range.

FIG. 12B is a side-view of an embodiment of a probe with range finding capability while imaging the cervix.

FIG. 13A is an isometric-view of an embodiment of a probe with a spacer rod with oximetry sensors.

FIG. 13B is a schematic diagram showing the probe of FIG. 13A inside the vagina.

### DETAILED DESCRIPTION

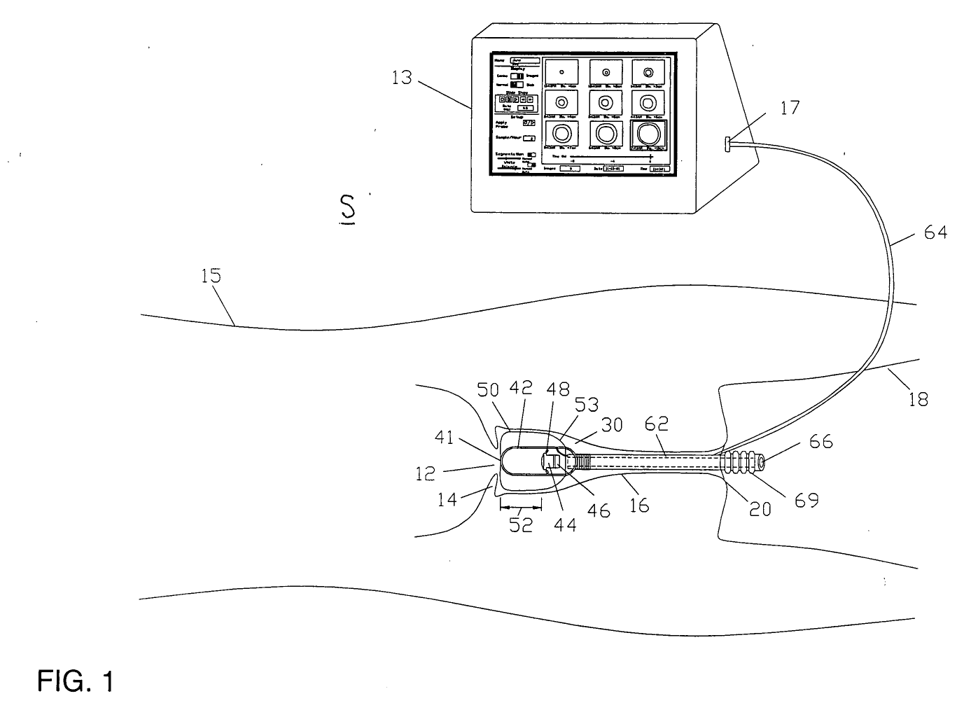

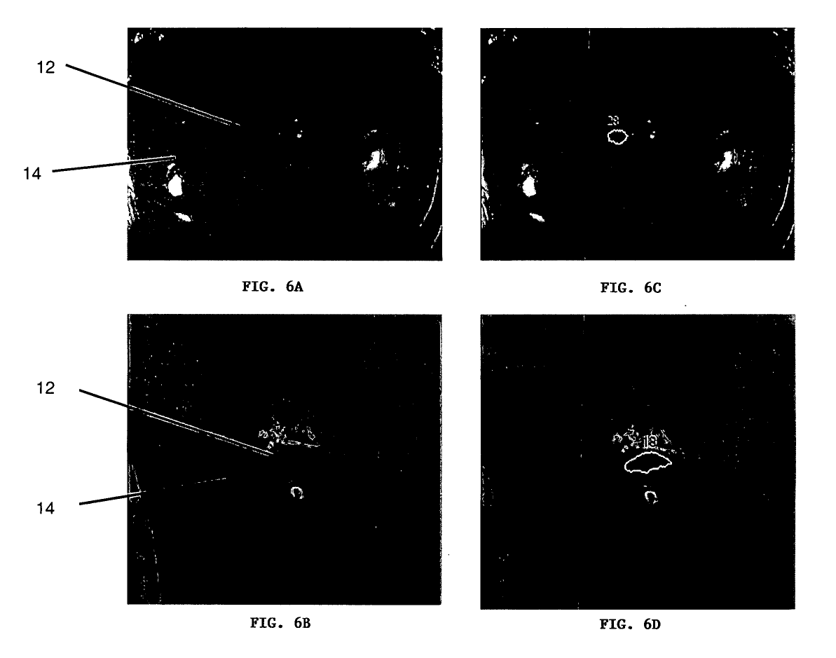

Referring to FIGS. 1 and 6, the present invention includes a system S for monitoring dilation of an opening 12 of a cervix 14 of a female patient 15 who is pregnant and in labor to deliver. The system includes a probe 30 that is inserted into a vagina 16 of the patient to gather data relating to the cervix, in particular, the diameter of the opening, and a monitoring unit 13 that is in communication with the probe and receives data from the probe which is processed by a processor to provide and display useful information to health care providers located proximately to the patient and/or remotely therefrom. Advantageously, the probe 30 is adapted to remain in the patient's vagina throughout the duration of labor and assume a suitable position in the vagina so as to gather image data that is sent to the monitoring unit. Accordingly, the patient need not have her cervix digitally probed during labor and may remain relatively undisturbed while changes in the size of the cervix opening are monitored continuously or intermittently as appropriate.

Disposable Probe

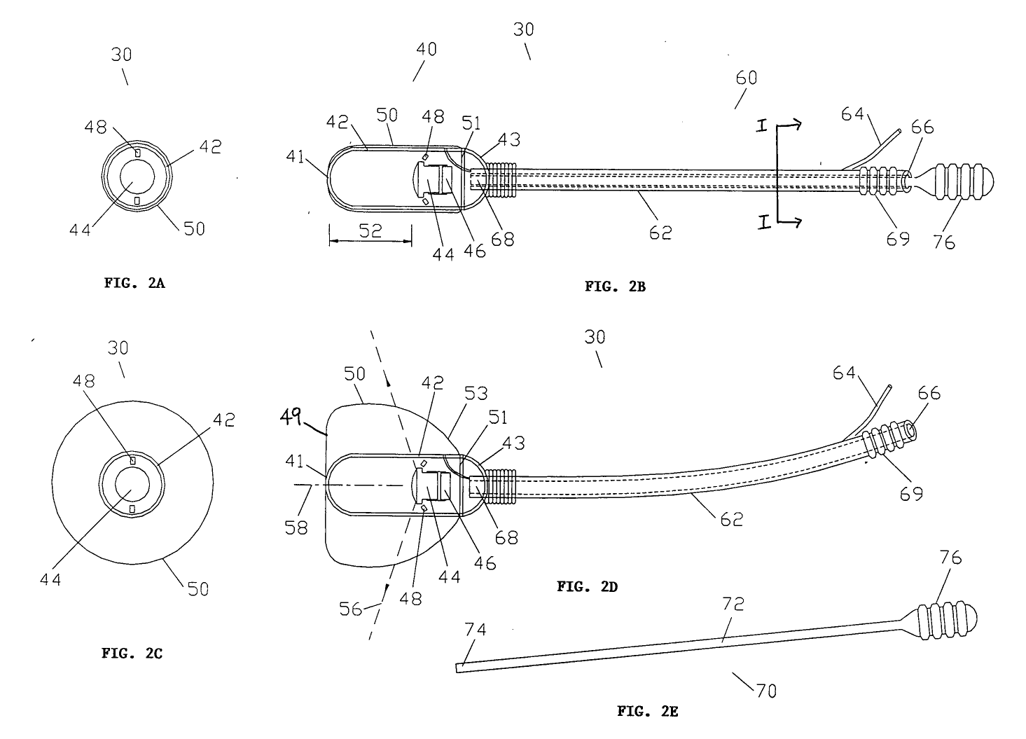

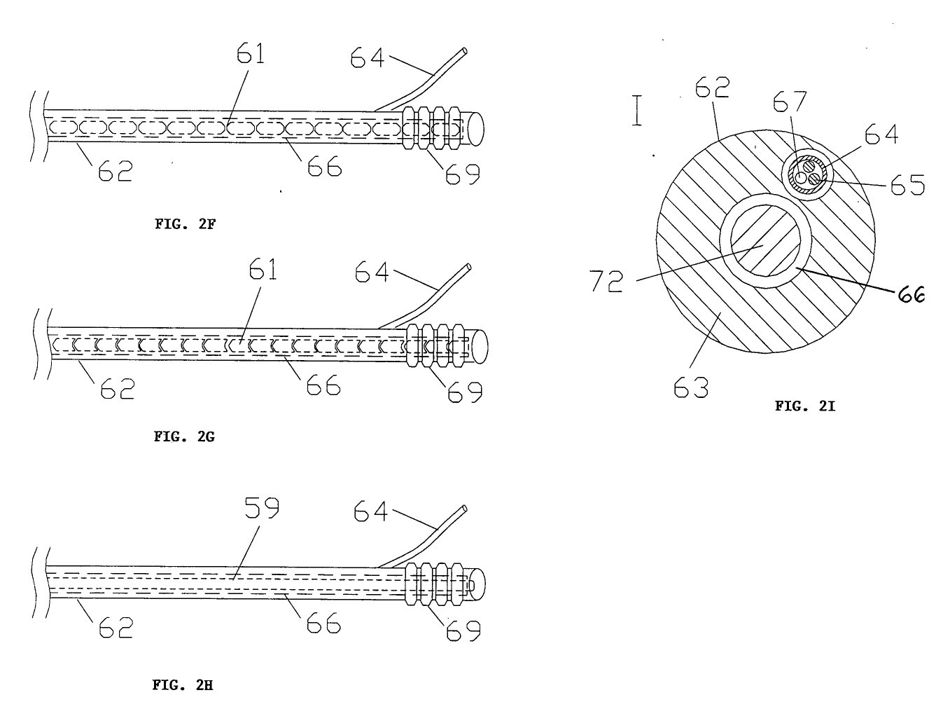

Referring to FIGS. 1 and 2A-2D, a preferred embodiment of the cervix probe 30 includes generally a distal probe head 40 and a proximal probe tail or handle 60 extending proximally from the probe head. The probe head 40 includes a housing 42 encapsulating a lens 44 and a camera 46 that is proximal of the lens and adapted to capture image data of objects within a predetermined field of view 56 of the lens. The housing 42 also contains at least one light source 48 positioned to illuminate the field of view of the lens inside the vagina. The lens 44, the camera 46 and the light source 48 are fixedly mounted within the housing to the proximal end 53 of the housing. Covering the housing is an inflatable cover or balloon 50 that is sealed to the housing along a circumference 51 at a proximal end portion of the housing 42. Adhesive and/or a ring clamp may be used to seal the balloon to the housing.

The probe handle 60 includes a flexible hollow tube 62 having a wall 63 through which a cable 64 extends proximally from the probe (FIG. 21) and exits at or near a proximal end of the tube. Extending through the cable are electrical wires 65 extending from the probe 30 to the monitor unit 13 and a fine tube or air conduit 67 whose distal end extends into the housing 42 and terminates at or in the balloon 50 for air or fluid communication therewith.

The housing 42 may be generally cylindrical in shape along a longitudinal axis 58 with preferably a streamlined distal and proximal ends 41 and 43 to facilitate insertion into and removal from the vagina 16. Alternatively, the housing may be shaped as the frustum of a cone with a wider distal portion and a narrower proximal portion. The housing has a length in the range of about 2 cm to 5 cm, and more preferably about 4 cm. The housing has a diameter of about 1 cm to 3 cm, and more preferably about 2 cm. The housing 42 may preferably be made of an optically transparent plastic material such as, for example, polycarbonate; however, other materials such as Pyrex may be used. The housing 42 may be hermetic to protect its internal components from contamination and the outside environment. The housing 42 may also be coated or made of a hydrophobic (i.e. water repellent) material to prevent fluids, if any, from adhering to its exposed surfaces. The housing 42 may also have an intermediate depression to accommodate the balloon 50 when deflated.

The camera 46 is of the miniature type with a size ranging between about 16×16×8 mm and 6×6×3 mm and more preferably 8×8×4 mm. The lens 44 may be a wide angle lens, its field of view (FOV) 56 ranging between about 120 to 190 degrees and more preferably about 170 to 180 degrees to enable imaging of the cervix 14 from a relatively close range. In that regard, the range is generally provided by a predetermined distance or separation 52 between the lens 44 and the distal end 41 of the housing which is also generally the distance between the lens 44 and the cervix 14, since the probe 30, as described below in further detail, is adapted to position itself in the vagina with the distal end 41 of the housing generally abutting the cervix or the cervix opening. Accordingly, the camera 46 and the lens 44 are selectively and fixedly situated within the housing 42 at the distance 52 from the distal end 41 of the housing 42. In particular, the distance 52 is selected to provide enough range to allow the field of view 56 of the lens 44 to capture a fully dilated cervix (at about 10 cm or so) when the distal end 41 of the housing 42 is touching, or is proximate to, the cervix 14 or the opening 12. The distance 52 may range between about 1 cm to 2.5 cm, and more preferably about 2 cm. The camera 46 may be of any type, including color, grayscale, CCD, CMOS, analog, digital, multispectral, or thermal. Optical filters may also be used to remove certain wavelength bands to enhance the image and/or clarify features of interest such as the opening 12 of the cervix 14.

One or more light sources 48 are disposed around the camera to provide the proper illumination necessary for imaging without saturating the camera 46 by, for example, internal reflections off the inner surface of the balloon 50. The light sources 48 may be light emitting diodes (LEDs) of the miniature surface mount type (SMD), bulbs, or optical fibers. The optical fibers, if used, may have a tip that is polished at an angle to provide side emission. The light sources 48 may emit white or monochromatic light at certain wavelengths, including infrared, to provide better viewing of different tissues/materials, glare reduction and/or improved imaging. The light sources 48 may be aimed at different angles and may be illuminated simultaneously, individually and/or in groups to improve imaging and/or image quality and avoid saturation of the camera 46.

The balloon 50 may be made of an optically transparent material such as silicone, latex, polyethylene terephthalate, polyurethane, and/or other materials. The material of the balloon 50 may or may not be elastic. The balloon 50 may be collapsed passively under its own elasticity, and/or actively by suction through the conduit 67 and/or compression from surrounding vaginal tissue. During assembly of the probe 30, the balloon 50 may be snugly fitted over all or a portion of the housing 42 as shown in FIG. 2a and FIG. 2b, with its end sealed to the housing at the circumference 51. As such, the deflated balloon 50 may assume the shape of the housing as shown in FIG. 2a and FIG. 2b. A portion or portions of the balloon 50 may be attached directly or tethered to the distal end 41 and the proximal end 43 of the housing 42 such that the housing 42 is suspended within the balloon when it is inflated. The inflated balloon 50 is configured such that it may preferably assume a conical shape (flask-shape) that is coaxial with the housing 42, with a cone base 49 at the distal end 41 and generally a cone apex 53 at the proximal end 43 of the housing 42 as shown in FIG. 2d. The advantages of this slightly conical inflated balloon shape include: (1) facilitating removal and retrieval of the probe 30 from the vagina 16 even with the balloon inflated, (2) providing maximum vaginal widening close to the cervix 14, and/or (3) urging of the probe 30 to position itself toward the cervix 14. Accordingly, upon inflation of the balloon 50, the distal end 41 of the housing 42 generally borders and axially aligns itself with the cervix 14 and/or the opening 12 to approximately establish the range distance 52 between the lens 44 and the cervix 14 as shown in FIG. 1. Advantageously, the conical shape of the probe 30 is adapted to impede sliding proximally in the vagina, toward the vulva 20, while facilitating sliding toward the cervix 14. Moreover, to limit sliding of the probe proximally toward the cervix 14 (which may allow the probe to become misaligned with the cervix), the cable 64 extending from the vagina may be fixed to the thigh 18 of the patient 15 using adhesive tape to tether the probe to the thigh. Furthermore, as mentioned, the conical balloon shape with its apex 53 directed toward the vulva 20 also facilitates the removal of the probe 30 from the vagina 16 even with the balloon 50 inflated. In addition, the conical shape of the inflated balloon 50 better conforms to the shape of the inner portion of the vagina 20 during labor.

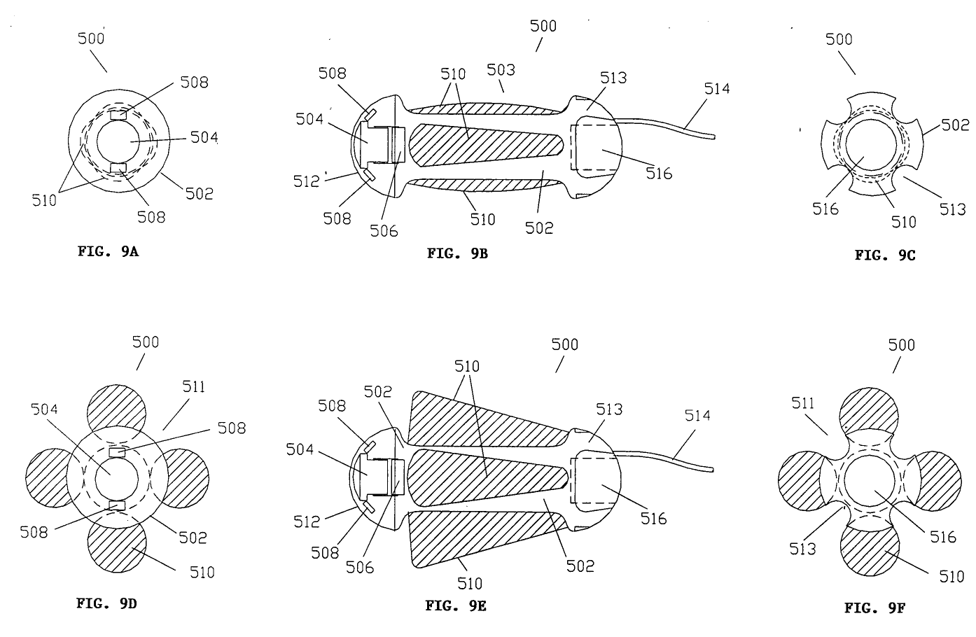

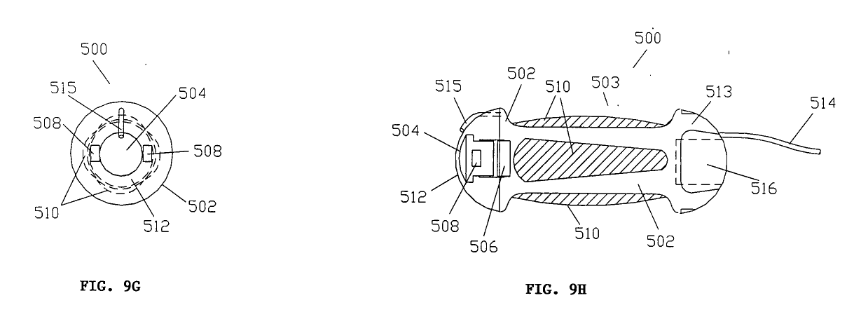

Alternatively, the balloon 50 may assume any other generally axially symmetrical shape when inflated provided it avoids distorting or obstructing the field of view 56 of the lens 44 and the camera 46. Other shapes of the balloon 50 include spherical, elliptical, egg-shaped, cylindrical, ribbed-cylinder, star, and cuboidal. Yet alternatively, the balloon 50 may be multi-compartmental and/or composed of two or more inflatable chambers 510 that may be inflated either simultaneously, individually, or in groups using separate tubes to control the spatial orientation of the probe 30 within the vagina 16 (see FIGS. 9A-9F). Inflation and deflation of the balloon via the conduit may be automated by the processor as part of image processing algorithms. For example, the processor may continue to inflate the balloon until the image processing algorithms can identify the opening of the cervix in images acquired by the camera 46.

The balloon 50 is attached to the housing 42 such that it expands symmetrically about the axis 58 to provide the lens 44 and the camera 46 with a centered view of the opening 12 of the cervix 14. Alternatively, the balloon 50 may be attached to the housing 42 such that it expands asymmetrically about the axis 58 of the housing 42 when inflated to obtain an off-centered view of the opening 12 of the cervix 14. The latter configuration may help minimize any optical effects or distortion of the distal end of the housing. In any case, any optical effects or distortion in the image data caused by the distal end 41 may be compensated for and corrected by the image processing algorithms.

The probe handle 60 includes a generally flexible tube 62 emerging proximally from the proximal end 43 of the housing 42. The tube 62 may be made of silicone, latex, Tygon or any other flexible tubing material. The outer diameter of the tube 62 is no greater and preferably smaller than the diameter of the housing 42 as shown in FIGS. 2B and 2D. The tube 62 is preferably long enough to allow it to exit the vagina 16 when the distal end 41 of the housing 42 is in contact with the cervix 14. A suitable length ranges about 10 cm to 18 cm and more preferably about 12 cm to 14 cm. A wall of the tube 62 may be configured with an aperture to enable the cable to exit at or near the proximal end of the tube and extend to the monitoring unit 13. It is understood by one of ordinary skill in the art that the electrical connection between the probe 30 and the monitoring unit 13 by which image data and/or control signals are sent and received need not be accomplished by wires but that it can be wireless as well, or a combination of the two.

In accordance with the present invention, the handle 60 provides several advantages, including: (1) preventing a total collapse of the vagina 16 proximal to the probe head 40 so as to minimize vaginal irritation upon sudden withdrawal of the probe 30 from the vagina, (2) allowing convenient removal, adjustment, and reinsertion of the probe 30, (3) providing stabilization and centering of the probe 30 with minimal discomfort to the patient 10, and/or (4) protecting the cable 64 of the probe. In another embodiment of the handle 60, the wall of the tube 62 may be configured along its length with drainage holes (not shown) to facilitate seepage into the tube of any fluids present in the vagina 16 and drainage of the fluids out of the proximal end of the tube.

The probe handle 60 may include an applicator or shaft 70 to reinforce the flexible tube when the probe 30 is inserted into the vagina 16. The applicator 70 is generally a rod 72 that is inserted into a lumen 66 of the tube and advanced distally until its distal tip 74 engages a docking hole 68 configured in the proximal end 43 of the housing 42. After the probe 30 is inserted into the vagina 16, the applicator 70 is drawn from the lumen 66 to restore the flexibility of the tail 60. A proximal end of the applicator has a gripping knob 76 to facilitate handling of the applicator 70 and/or the probe 30 by a health care provider.

In an alternative embodiment as shown in FIGS. 2F and 2G, the lumen 66 may permanently house a train of beads 61 to provide the appropriate structural support to the tail 60 when being inserted into the vaginal while maintaining the flexibility of the tail to minimize post application discomfort. The beads may be preferably semi-cylindrical as shown in FIG. 2F and/or semi-spherical and preferably hollow to reduce their weight. The beads may have articulating ends such that a protrusion from one bead would articulate in a socket in adjacent bead(s) and so forth as shown in FIG. 2G. Moreover, the beads may be threaded with a tension string that can be used to temporarily compress the beads and convert them to a stiff rod only during the insertion of the probe 30. Yet alternatively, the lumen 66 may include an inflatable body 59 as shown in FIG. 2H that may be temporarily inflated by air or liquid to reinforce the tail 60 and facilitate the insertion of the probe 30 into the vagina 16. The inflatable body 59 may be inflated or deflated via a dedicated air or fluid conduit in the cable 64.

The cervix probe 30, even when inflated, can be removed relatively quickly from the vagina 16 by manually holding grip treads 69 on the proximal end of the tube 62, which is preferably extending outside of the vagina, and pulling the probe 30 proximally The cable 64 may have an inter-connector member (not shown) that is adapted to connect with an extension cable (not shown) connected to the monitoring unit. This configuration may allow a shorter cable to be used with the probe.

Reusable Probe

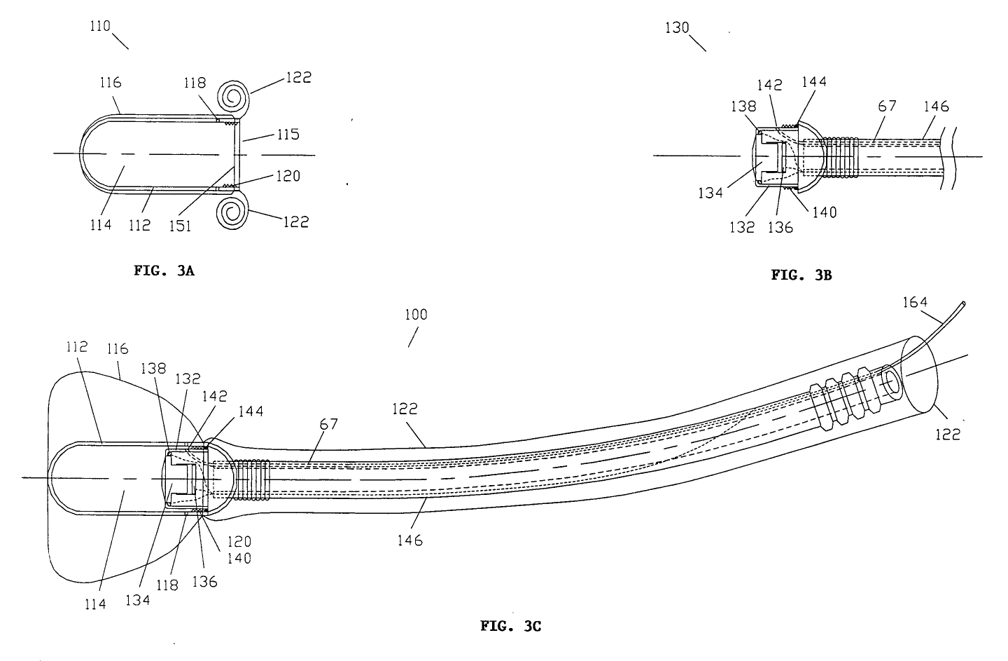

FIGS. 3A, 3B and 3C show an alternative embodiment of the probe which provides disposable (typically less costly) components and reusable (typically more costly) components. A probe 100 as shown in FIG. 3A has many structural similarities to the probe 30 of FIG. 2; however, the probe 100 is configured with a cap 110 that is disposable and a core 130 that is reusable, where the cap and the core are releasably coupled to each other. In one embodiment, the housing is divided into a cap housing 112 (FIG. 3A) and a core housing 132 (FIG. 3B). The cap housing 112 defines an interior 114 accessed via an opening 115 that is adapted to receive a distal portion of the core 132. The cap 110 also includes a balloon 116 covering the cap housing 112, and a condom-style protective sheath whose distal end is attached to the cap housing at the circumference 151 and whose proximal portion 122 is rolled up at or near the opening 115 of the cap housing 112 which has an interior threaded portion 120. The cap housing is also configured with an aperture 118 through its wall to provide a passage way for air to inflate the balloon 116, which is similarly sealed at the circumference 51 (see FIG. 1), as well as exit from the balloon when deflating.

The core 130 shown in FIG. 3B is composed of the core housing or base 132 on to which are fixedly mounted a lens 134, a camera 136 proximal of the lens and a light source 138 generally adjacent to the lens to illuminate a field of view of the lens. The core housing 132 is configured on its exterior with a threaded portion 140 suitable for releasably coupling the core housing 132 to the cap housing 112. The core 130 also includes an air outlet 142 adapted for communication with the air passage way 118 at one end and with the conduit 67 (FIG. 2B) at the other end, and a gasket 144 to seal the coupling between the core 130 and the cap 110. All or part of the cap housing 112 and the core housing 132 may be optically transparent to allow a relatively unobstructed field of view for imaging the cervix 14.

Prior to inserting the probe 100 into the vagina, the probe is assembled by placing the cap 110 over the core 130 and connecting the cap and the core to each other, for example, by the threaded portions. As understood by one of ordinary skill in the art, the use of screw thread is not restrictive and other locking mechanisms to releasably couple the core and the cap, for example, a snap fit configuration, may be used. The gasket 144 seals the interior 114 between the cap 110 and the core 130. The distal end of the air conduit 67 is in communication with the air outlet 142 of the core housing 132 which is in communication with the distal portion of the balloon via the aperture 118. There may be additional apertures in the housing 112 that permit air to pass between the interior 114 and the balloon 116. As described before, air is pumped through the air conduit 67 to reach and inflate the balloon 116 and air is expelled from the balloon through the air conduit 67 when deflating the balloon. The above pneumatic means of connecting and communicating air to and from the balloon is not restrictive and other configurations may be used. For example, in an alternative embodiment, the pneumatic connection may be accomplished by a male tube in the core 130 that plugs into a female receptor hole in the cap 110 to establish pneumatic continuation upon placement of the cap 110 over the core 130. In any case, after attaching the cap 110 to the core 130, the proximal portion of the protective sheath may be unrolled and deployed over the core 130 and the tail 146 as shown in FIG. 3c to cover and protect them from contamination by fluids or debris in the vagina 16. The proximal portion 122 of the protective sheath is of a length to adequately cover the tail 146 and cable 164 to outside of the vagina 16 and preferably by a sufficient length beyond the vulva 20. The cable 164 may be taped to the thigh 18 of the patient 15 to prevent any unintended dislodging of the probe 100 from the vagina.

Following use of the probe 100, the core 130 is decoupled from the cap 110 and the cap 110 is discarded or otherwise disposed of. The core 130 on the other hand may be sterilized using common medical detergents and/or cleaning agents for reuse with another cap. Other features of the probe are similar to the disposable probe 30 of FIG. 2 and generally the same specifications and functions apply.

The Monitoring Unit

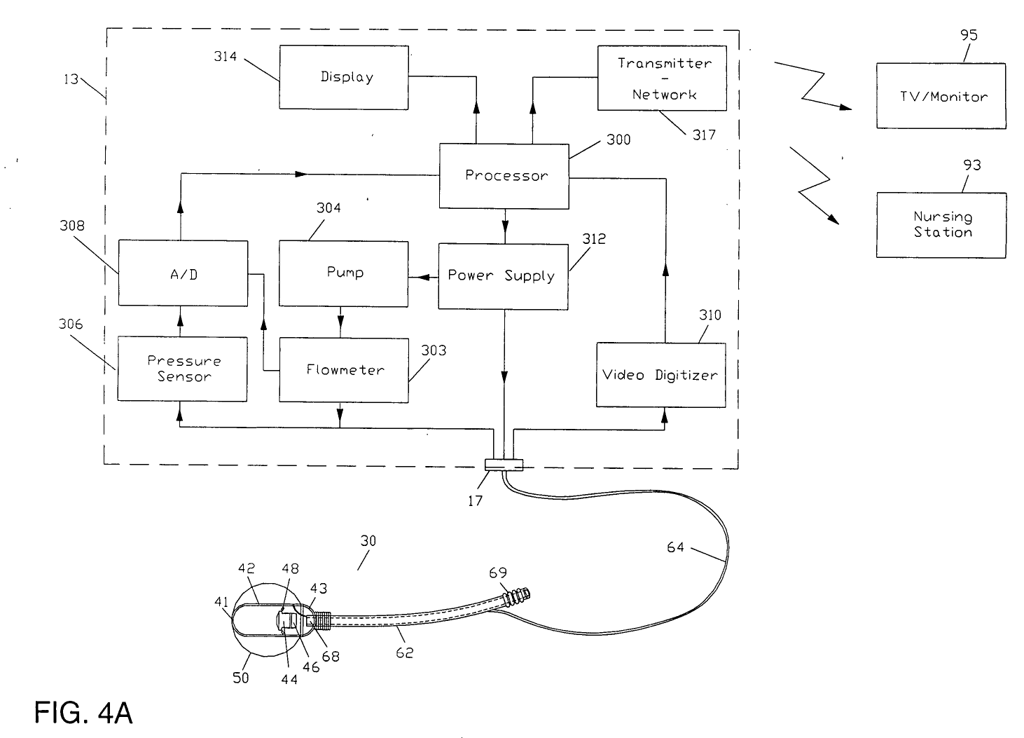

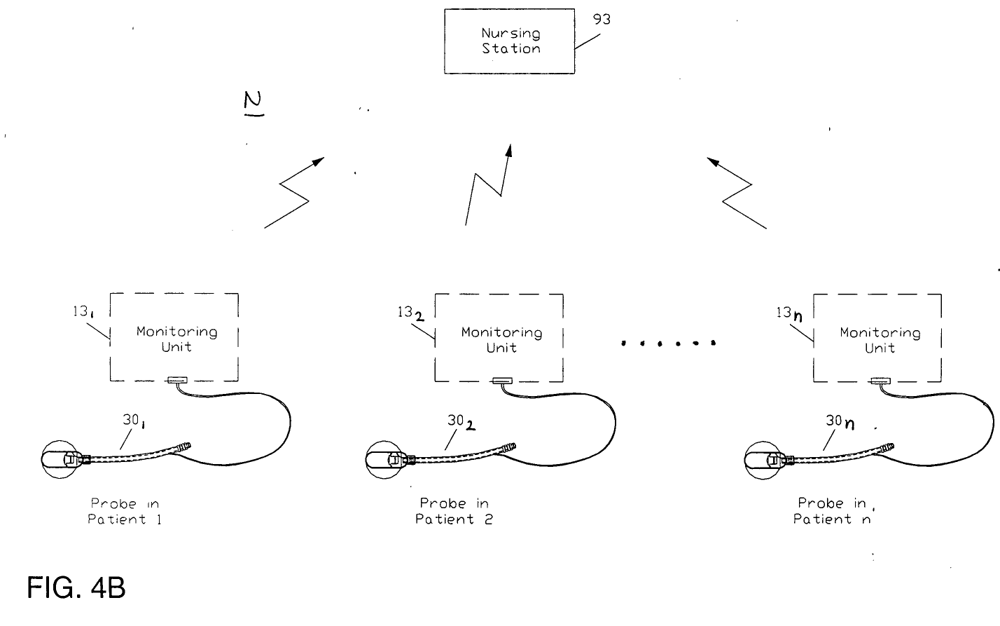

Referring to FIG. 4a, a block diagram of an embodiment of a system of the monitoring unit 13 is shown in use with the probe 30. The unit 13 has a power supply 312 supplying power to the system S, a pump 304 for supplying air to and drawing air from the balloon 50 of the probe 30, a flowmeter 303 to monitor the flow into and out of the balloon, a pressure sensor 306 to monitor the pressure of the balloon, a video digitizer 310 that digitizes the image data from the probe 30, a processor 300 that controls the operation of the probe 30 and the system S, and an image display 314. The processor receives and processes the image data provided by the probe, including an implementation of the image processing algorithms that process the image data provided by the probe, and sends data signals to the image display, which displays information relating to the changes in the cervix opening as an indicator of the progression of labor.

A cable connector 17 facilitates releasable connection of the probe cable 64 to the monitoring unit M. The cable connector 17 includes both electrical connectors and tube connectors. The pump 304 may be a low-noise bidirectional air pump or a bidirectional liquid pump to pump saline or any other suitable fluid to inflate the balloon 50. The pressure sensor 306 may be used to measure pressure in the balloon 50 and control the pump 304. It is understood by one of ordinary skill in the art that the pressure sensor 306 may be situated in the monitoring unit 13 as shown in FIG. 4a, or alternatively in the probe 30 and connected to the monitoring unit 13 using wires in the cable 64. If the pressure sensor 306 is situated in the monitoring unit 13, a special control protocol may be required to measure a true air pressure in the balloon.

During pumping, the pressure measured at the pump 304 may not be equal to the pressure in the balloon 50 because of the airflow resistance of the air conduit 67 connecting the pump 304 to the balloon 50. As such, the pressure in the balloon may be measured using an alternating pump activation, deactivation, and pressure measurement protocol as follows: (1) The pump is activated for a period T1 and then deactivated, (2) A period T2 is provided such that pressure between the balloon 50 and the output of the pump 304 to which the pressure sensor 306 is connected is permitted to equalize, (3) The pressure is measured at the output of the pump 304, (4) The pump is activated again and so forth until the desired pressure is reached. The period T1 may be dynamically varied depending on an expected incremental increase in pressure. For example, T1 may be shortened as the pressure increases.

The flow meter sensor 303 measures the air volume pumped into or out of the balloon 50. This air volume information may be used along with or separate from the pressure information measured by the pressure sensor to control the pump until a desired volume is moved into or out of the balloon 50. The air volume to be pumped into the balloon may be adjusted depending on the latest measured diameter of the opening 12 of the cervix 14. The air volume to be pumped out of the balloon may be equal to the last air volume pumped into the balloon. The processor 300 may use an analog to digital converter 308 to acquire the measurement of the flowmeter 303 and the pressure sensor 306.

The video digitizer 310 may be used to digitize the video signal from the camera 46 of the probe 30 and convert it to a digital image for processing by the processor 300. Alternatively, a camera controller (not shown) may replace the video digitizer 310 where the camera is of the digital type. The power supply 312 may be used to power the components of the cervix probe 30 and the monitoring unit 13.

In one control sequence, a switch (not shown) in the cable connector 17 may trigger the processor 300 when the probe 30 is plugged into the monitoring unit 13. The processor 300 automatically powers up the camera 46, the light source 48, the video digitizer 310 and the display 314 to provide live video guidance to aid the user in positioning the probe 30 in the vagina 16 (e.g. next to the cervix). Alternatively, the probe 30 may be blindly inserted into the vagina 16 until the user determines that it cannot be advanced any further. At this position, the distal end 41 of the housing 42 should be touching the cervix 14. After the probe 30 has been appropriately positioned in the vagina 16, the processor 300 may begin to acquire measurements using the following cycle:

1. The processor 300 may activate the pump 304 to inflate the balloon 50 up to a volume and/or pressure level that is predetermined to sufficiently expand the vagina for proper imaging of the cervix 14.

2. The processor 300 may activate the camera 46, the light source 48, and the video digitizer 310 to capture an image of the cervix 14 and its opening 12.

3. The processor 300 may reversibly activate the pump 304 to deflate the balloon 50 to the pre-inflation volume and/or pressure.

4. The captured image may be initially preprocessed to correct for any optical distortions (e.g. barrel distortion) that may be caused by the wide-angle lens 44, the housing 42 and/or the balloon 50.

5. The distortion-corrected image is processed using image-processing algorithms as described below to identify the cervix opening 12, measure its diameter, and calculate the dilation rate from any sequential measurements.

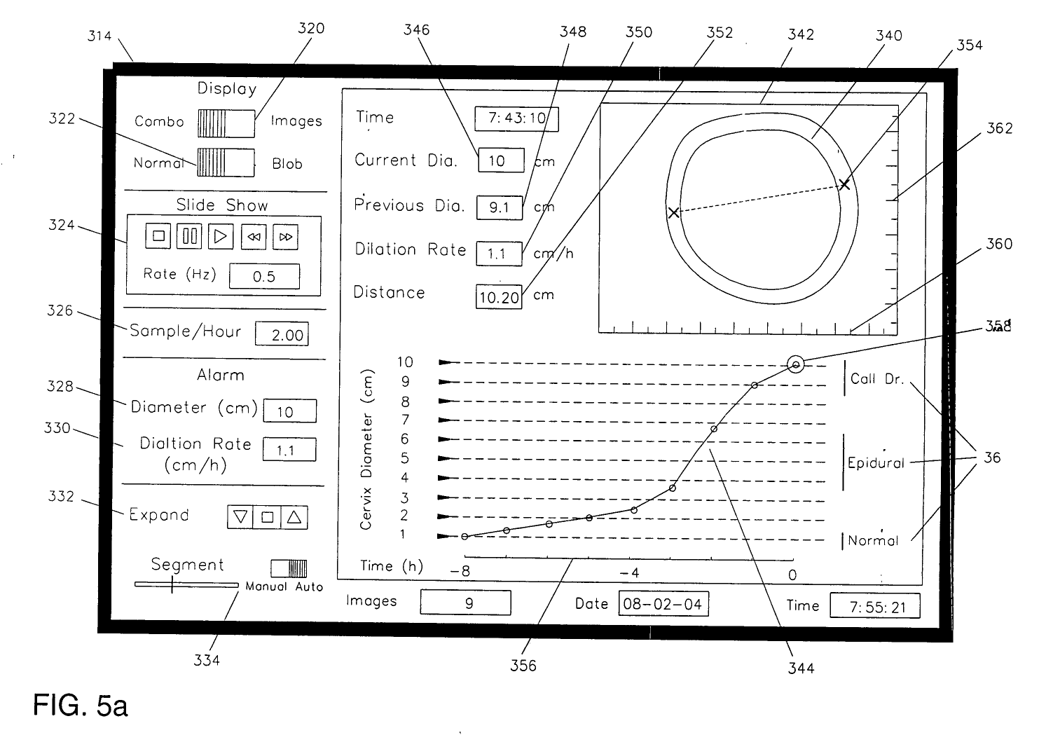

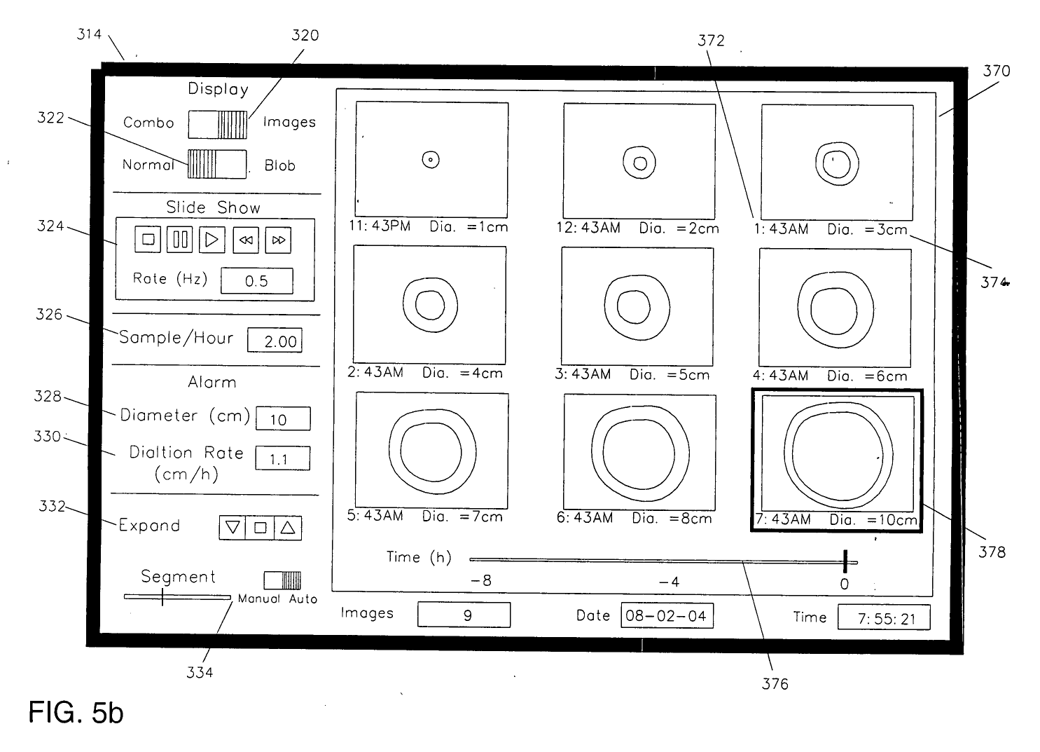

6. The captured image is displayed on the display 314 along with, for example, its corresponding diameter of the cervix opening 12 and the dilation rate. Examples of preferable display modes are shown in FIG. 5.

The above data acquisition cycle is not mandatory and other operation sequences may be used if desired, as understood by one of ordinary skill in the art. The data acquisition cycle may be repeated every 30-minutes or at any different rate that is selected by the user.

The display 314 may be of the touch-screen type and may be divided into a control section 315 and a data display section 317. The control section allows the user to select operating parameters. The user may use a graphical switch 320 to select between two data display modes, for example: (1) a Combo mode as shown in FIG. 5a or (2) an Images mode as shown in FIG. 5b. Also, the operator may use the graphical switch 322 to select a view of normal images or a view of segmented images with the opening 12 of the cervix 14 graphically outlined. Slide show control buttons 324 allow the user to activate a series or slide show sequencing the images of the cervix 14 one after the other in the display section. The operator may also: (a) use a window 326 to select a cervix probing rate in units of samples per hour, (b) use a window 328 to select an alarm level to warn when the opening of the cervix reaches a certain diameter, (c) use a window 330 to select an alarm level to warn when the dilation rate is abnormally slow relative to the stage of labor and/or the diameter of the cervix opening 10, (d) use a graphical button 332 to manually control the inflation/deflation of the balloon 50 and/or (e) use a graphical button and slider 334 to perform manual segmentation of the initial cervix image. The parameters setting of this initial manual segmentation may be applied to automatic segmentation of subsequent images.

The display section of the Combo Screen shown in FIG. 5a displays generally: (1) a cervix image 340 in the window 342, (2) a cervix dilation graph 344 plotting the diameter of the cervix opening 12 versus time, and/or (3) a set of numerical values, including the automatically measured diameter of the cervix opening 346, the previous diameter measurement 348, the dilation rate 350, user-measured distance 352 obtained by positioning two cursors 354 on the cervix image 340. The Time “0” on the time axis 356 of the graph represents the latest (or current) measurement while negative values represent past time. For example, the “−4” indicates four hours earlier. The user may use a pointing device to position two cursors 354 on the cervix image 340 to measure any desired distance and display the resulting value window 352.

Each point in the cervix dilation graph 344 may be clicked on or otherwise selected to display its corresponding image of the cervix opening in the window 342. The circle or marker 358 highlights the data point associated with the cervix image 340 displayed in the window 342. The window 342 displaying the cervix image 340 may have a horizontal ruler 360 and a vertical ruler 362 to allow the eyeballing of physical dimensions (i.e. cm). A grid may be superimposed on the window 342 if desired. Text comments 364 are possible prompts or suggestions of actions that may be taken when a specific diameter is reached.

The Images Screen shown in FIG. 5b generally displays a series of images 370 showing the cervix 14 as it dilates throughout labor. Each image is stamped with: (1) a time of its acquisition 372, and (2) an automatically measured diameter of the cervix opening 374. The sliding indicator 376 gives a graphical impression of the temporal location of the image highlighted by the frame 378.

The monitoring unit 13 may include a transmitter 317 to transmit the acquired images of the cervix and other corresponding information including the cervix diameter and dilation rate to a remote station, such as a nursing station 93 and/or a television 95. The transmitted signals may be transmitted on different frequencies or digitally coded to prevent the interference of signals upon reception. The transmitted signals may be also encrypted for security purposes. The monitoring unit 13 may also include a receiver (not shown) for bidirectional communications to receive operation instructions from the nursing station 93. Clearly, it is understood by one of ordinary skill in the art that multiple systems S₁-S_{N}, with multiple units 13₁-13ₙ and their respective probes 30₁-30ₙ, can be configured to communicate with each other and/or one or more nursing stations as a monitoring network for monitoring different patients as shown in FIG. 4b.

Image Processing

The captured image of the cervix may be preprocessed to correct for any spatial distortion caused by the wide-angle lens 44 of the camera. The wide-angle lens 44 (i.e. fisheye) (FIG. 1) may be used to enable the imaging of a fully dilated cervix 14 from a relatively close distance of approx. 2 cm. Wide-angle lenses can introduce an image distortion known as the barrel distortion, which is caused by the uneven magnification between the edges and the center of the lens. Barrel distortion is a type of radial distortion in which horizontal and vertical lines appear to be bent outwards toward the edges of the image. Algorithms to correct barrel distortion in images are readily available in the literature, e.g., Mundhenk, T. N., et al., “Techniques for fisheye lens calibration using a minimal number of measurements,” Proceedings of the SPIE, SPIE-Int. Soc. Opt. Eng., 4197, pp. 181-90, 2000, and e.g. James P. Helferty, et al., “Videoendoscopic Distortion Correction and Its Application to Virtual Guidance of Endoscopy,” IEEE Transactions on Medical Imaging, Vol. 20, No. 7, pp 605-617, 2001. These algorithms may be applied to the images captured by the video digitizer 310 (FIG. 4a) to minimize or remove barrel distortion. In addition, algorithms for the correction of perspective distortion (e.g. Waltz, F. M., “Implementation of real-time perspective correction,” Proceedings of the SPIE—SPIE-Int. Soc. Opt. Eng. 849, pp. 179-83, 1988) may be also applied to correct for distortions caused by the non-perpendicular imaging of the cervix (i.e. when the probe is at a tilted viewing angle of the cervix). The distortion-corrected images may be color balanced and filtered using, for example, a median filter to improve image quality.

Moreover, images of the cervix acquired by the probe may be visually contaminated by the presence of biological fluids, mucus, etc., on the cervix during its imaging. This may require special preprocessing to enhance the image quality before applying the segmentation algorithms for the identification and measurement of the cervix opening. One method to improve image quality may be to average images captured under different illumination settings (e.g. angle, intensity or color). Another method may be to use images captured from two or more separate balloon inflations. This may be achieved by inflating the balloon, capturing a first image, slightly deflating the balloon, immediately re-inflating the balloon to capture a second image. The first and second images may be used individually to measure the diameter of the cervix; e.g., the two measured diameters are averaged to determine the actual diameter and/or an effective diameter of the cervix opening. Alternatively, the first and second images may be averaged and the resulting image used to measure the diameter of the cervix opening.

The processor 300 may use image segmentation techniques and/or blob analysis algorithms to analyze the images of the cervix 14 to identify its opening 12 and measure its diameter. Blob analysis is a branch of image analysis that allows the identification of connected regions of pixels (known as blobs) within an image. Once these regions are identified, one can calculate selected features of those regions, automatically discard regions that are not of interest, and classify the remaining regions according to the values of the features. A blob region may be identified by segmenting the image such that the pixels of an object have the same logical state. Regions of touching pixels in this state are identified as a blob. Pixels not part of a blob may be considered as image background.

Segmentation is a well-known image processing method that is used to isolate a desired image features or object from background of the image. It is commonly used in blob analysis, machine vision and medical imaging. In this case, object segmentation is applied to isolate the opening of the cervix from the rest of the image. The segmentation techniques applied may include intensity thresholding, color thresholding, active contours, region growing, and/or level set analysis. The above methods are described in known literature, e.g., Pratt, William K., Digital Image Processing, John Wiley & Sons, New York, 2001, and e.g., Jahne, Bernd, Image Processing for Scientific Applications, CRC Press, New York, 1997, and may provide a reliable and accurate means to isolate and measure the opening 12 of the cervix 14. The area of the segmented objects within the image may be compared to preset values of minimum and maximum areas of the cervix opening to automatically discard objects with unrealistic dimensions or areas. The processor 300 may counts the number of pixels within the segmented opening of the cervix and converts the number of pixels to an area in real dimensions (cm²). The actual physical dimensions of each pixel are pre-known since the lens 44 of the camera 46 is separated by a known distance 52 from the cervix 14 using, for example, the probe embodiment described in FIG. 2. Assuming the cervix opening during labor is circular, the average diameter “D” of the cervix opening may be approximately calculated using the following formula:

$$D = \sqrt{\frac{4 Segmented Area [ {cm}^{2} ]}{π}} [ {cm}^{2} ]$$

The measured diameter value “D” may be compared to the previously measured diameter, if any, to calculate the dilation rate. The processor 300 then displays the preprocessed image of the cervix, the measured diameter, and dilation rate on the display 314.

Although the above image-processing techniques are described with one probe embodiment as an example, it should be appreciated that these image-processing techniques may be used with any of the alternative probe embodiments described herein.

FIG. 6a is a photographic image of an actual cervix with a generally centered cervix opening of a certain diameter. FIG. 6c is the image of FIG. 6a after it has been segmented and analyzed to identify the cervix opening and measure its area. The computer outlined area in FIG. 6c measures approximately 221 pixels.

FIG. 6b is photographic image of an actual cervix with a generally cervix opening of a greater diameter. FIG. 6d is the image of FIG. 6b after it has been segmented and analyzed to identify the cervix opening and measure its area. The computer outlined area in FIG. 6d measures approximately 522 pixels.

Since the lens has a known magnification, the above pixel areas may be converted into physical area and used in the above equation to calculate the approximate average diameter of the cervix opening.

Portable Controller

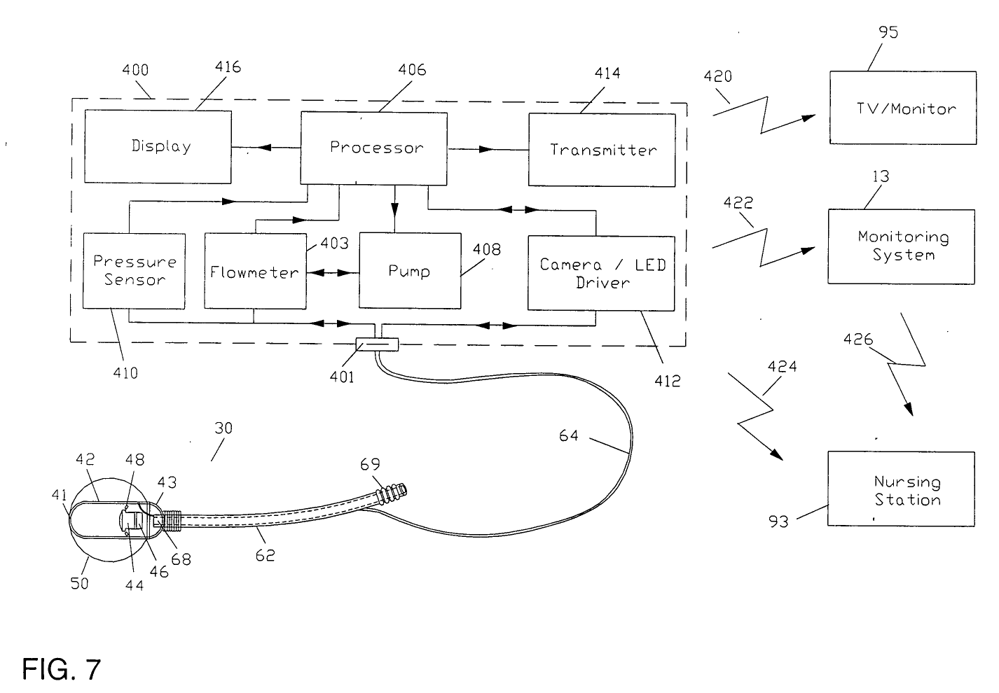

As another alternative embodiment, a portable wireless controller 400 may be used to facilitate the mobility of the patient 15 by eliminating the cable 64 connecting the probe 30 to the monitoring unit 13. The main components of a wireless controller 400 are shown in the block diagram of FIG. 7. The controller 400 includes a processor 406, a bidirectional air pump 408, a pressure sensor 410, a camera and light-source driver 412, and a transmitter 414 as shown in FIG. 7. The controller 400 is compact and may be attached to the thigh 18 of the patient 15 using a belt or any other means of attachment. The probe 30 may be connected to the controller 400 using a connector 401. The controller 400 may communicate wirelessly with the monitoring unit 13, a remote nursing station 93 and/or an image monitor such as a television 95.

In a typical cervix measurement sequence, the processor 406 triggers the pump 408 to inflate the balloon 50 up to a certain predetermined pressure that is monitored by the pressure sensor 410. The inflation of the balloon 50 widens the vagina 16 and allows the lens 44 to have an unobstructed view of the cervix 14. The processor 406 then powers the light sources 48 and the camera 46 to image the cervix 14 and/or the opening. The processor 406 transmits the images of the cervix 14 using the transmitter 414 to the corresponding monitoring unit 13, the nursing station 93, and/or the television 95 which may be located in the patient's room. Alternatively, the controller 400 may process the images of the cervix 14 to measure the diameter and calculate the dilation rate, for example, using an ASICS. The diameter and dilation rate may be displayed on an onboard display 416 and/or transmitted along with identifying information to the corresponding monitoring unit 13, the nursing station 93, and/or the television 95 in the patient's room.

The transmitter 414 may be of the analog or the digital type. The transmitted signals 420, 422 and 423 may be either transmitted on different frequencies or digitally coded to prevent the interference of signals upon reception. The transmitted signals 420, 422 and 423 may be also encrypted for security purposes. The controller 400 may also include a receiver (not shown) for bidirectional communications to receive operation instructions from the corresponding monitoring unit 13 and/or the nursing station 93.

Other Embodiments of the Cervix Probe

Capsule Balloon with Inflatable Frontal Spacer

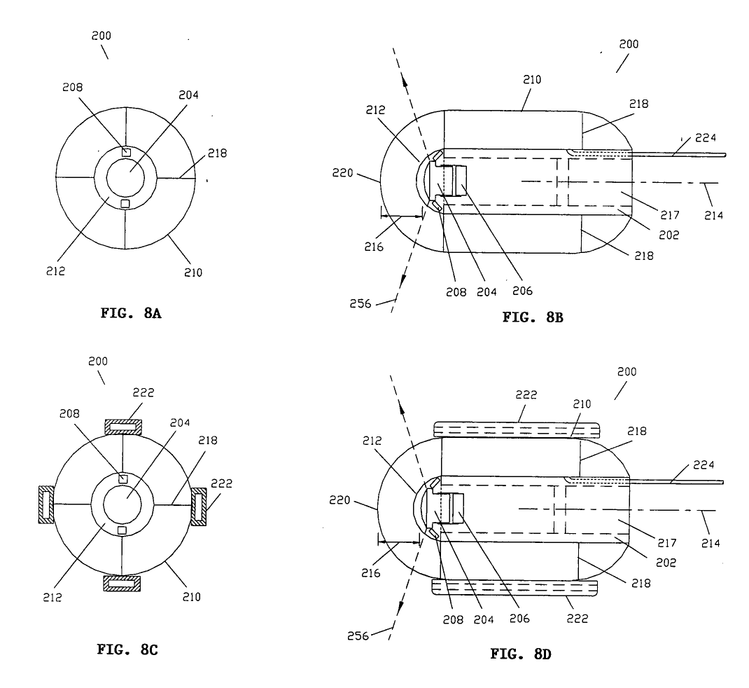

Another embodiment of a cervix probe 200 is shown in FIG. 8 where a balloon 210 expands into a capsule or bubble around the probe housing 202 to allow proper imaging of the cervix 14. The probe 200 is composed of a housing 202, a lens 204, a camera 206, and light sources 208, and the balloon 210. The lens 204 and the camera 206 may be positioned at the distal end of the housing 202 and has a field of view 256. The balloon 210 is positioned around the housing 202 such that it expands both radially and distally with respect to an axis 214 of the housing 202. The radial expansion opens the vagina to create an unobstructed field of view for the lens 204 and the camera 206 to image the cervix 14. The distal expansion creates a predetermined separation distance or range 216 between the lens 204 and the cervix 14 to: (1) provide enough range to allow the field of view 256 to capture a fully dilated cervix (at diameter about 10 cm or so) when the distal end 220 of the balloon 210 is touching, or is proximate to, the cervix 14 or the opening 12, and (2) maintain the dimensional calibration of the image by having a known distance between the lens 204 and the cervix 14.

The balloon 210 may be made of an optically transparent material such as silicone, latex, polyethylene terephthalate, polyurethane, or other materials. The material of the balloon 210 may or may not be elastic. The balloon 210 may be collapsed passively under its own elasticity or actively by suction. The balloon 210 may include support lines 218 (or tether ribbons) to hold the housing 212 in place and to limit distal expansion to maintain the separation distance 216 when the balloon is inflated.

The balloon 210 may be composed of multi-chambers or compartments that can be separately inflated for the independent control of the radial and distal expansions of the probe 200. Alternatively, two or more separate balloons may be used for the independent control of the radial and distal expansion of the probe 200. For example, first balloon may be used for radial expansion to widen the vagina while a second balloon may be used for distal expansion to create a known distance between the lens and the cervix.

With this embodiment, the cervix probe 200 may be placed in the vagina 16 such that a distal end 220 of the inflated balloon borders or touches the cervix 14 or its opening 12. The probe cable 224 may include wires and fine tubes to connect the probe 200 to the monitoring unit 13. The proximal end of the extension cable 224 has tube/wire connector 17, which plugs into the monitoring system 13. The proximal portion of the probe 200 may include a docking hole 217 where the distal end 531 of the applicator 530 (described below) may engage to hold the probe 200 prior to its application into the vagina 16. Alternatively, the probe 200 may include a handle similar to the handle 60 and use an applicator similar to the applicator 70 described above for the embodiment of probe 30.

Alternatively, radial surface of the balloon may have longitudinal draining tubes 222 arranged in radial symmetry about the axis 214 as shown in FIG. 8c and FIG. 8d. The longitudinal draining tubes 222 allow passage of any fluids proximally along the cervix probe 200 when the balloon is inflated so as not to occlude the vagina 16 and dam the fluids leaking from the cervix 14. The longitudinal draining tubes 222 may preferably have a rectangular cross-section, however, they may assume any other cross-sectional shape including circular, semicircular or square.

Multi-Balloon Conical Probe

Yet another embodiment of a cervix probe or probe module 500 is shown in FIGS. 9A-9F where the probe 500 includes a housing 502, a lens 504, a camera 506, and light sources 508. Multiple elongated inflatable bodies or balloons 510 are disposed radially about probe housing 502. The probe may have at least three inflatable bodies and, more preferably, at least four inflatable bodies disposed symmetrically about the probe housing. Front, side and back views of the preferred embodiment of the multi-balloon cervix probe 500 are shown in schematic diagrams of FIGS. 9A-9F.

The lens 504, the camera 506, and the light sources 508 are placed within a protective housing 502 and sealed from the outside environment. The protective housing 502 may be made of plastic and coated with silicone. The plastic and/or the silicone may be optically transparent. An optically transparent front shield 512 protects the lens 504 of the camera 506 and its surrounding light sources 508. The light sources 508 may be placed behind the shield 512 and optically coupled to the inner surface of the shield 512 to minimize back reflection into the camera 506. Alternatively, the light sources 508 may be placed on the outer surface of the shield 512, the housing 502, or the balloons 510 to provide optimal illumination of the cervix 14. The shield 512 is made of (or coated with) a hydrophobic (i.e. water repellent) transparent material to prevent fluids from adhering to its surface and obstructing the view of the camera. In addition, the front shield 512 may include an air nozzle 515 (FIGS. 9G and 9H) to emit short bursts of air (or saline) onto a front surface of the shield 512 to remove debris or fluids present thereon.

The streamlined housing 502 may be cylindrical and has smooth parabolic-shaped ends as shown in FIG. 9 to facilitate its insertion into and removal from the vagina 16. The outer surface of the housing 502 may have a shallow depression 503 to accommodate the balloons 510 when deflated. Each balloon 510 may preferably assume a conical shape when inflated. The balloons 510 may be arranged in a star configuration around the housing 502 such that the probe 500 may have a generally conical shape when the balloons 510 are inflated. An apex of the conical shape may be at a distal end of the probe 500 where the cable 514 exits. The generally conical shape of the probe 500 impedes movement or sliding distally, if any, out of the vagina 16 while enhancing movement or sliding proximally toward the cervix 14. Any sliding motion of the probe 500 toward the cervix 14 may be limited by fixing the cable 514 to the thigh 18 of the patient 15 using adhesive tape. The conical shape with its apex directed toward the vulva 20 also facilitates the removal of the probe 500 from the vagina 16 even with the balloons inflated. In addition, the conical shape better conforms to the shape of the vagina during labor.

The balloon 510 may be made of silicone, vinyl, latex, polyethylene terephthalate, polyurethane, or any biologically safe balloon material. The material of the balloon 510 may or may not be elastic. The balloon 510 may be collapsed passively under its own elasticity or actively by suction of the pump. The balloons 510 may be inflated either simultaneously or individually (e.g. using separate air tubes) to adjust the spatial orientation of the probe 500 within the vagina 16. The longitudinal space gaps 511 between the elongated balloons may serve as channels through which fluids can pass along the probe 500 and drain out of the body. A proximal portion of the body 502 of the probe 500 may include longitudinal channels (or grooves) 513 to facilitate the drainage of the fluids passing through the longitudinal space gaps 511.

After the cervix probe 500 is positioned at a selected location within the vagina 16, the balloons 510 are inflated to: (a) locally widen the vagina 16 and allow a better view of the cervix 14, and (b) releasably anchor the probe 500 at the selected location. A bedside monitor 13 may sense and adjust the pressure in the balloons 510 to maintain it at a minimum level that is needed to anchor the probe 500 and widen the vagina 16 for imaging the cervix 14.

A flexible cable 514 of the cervix probe may be attached to the patient's thigh 18 using an adhesive tape. The flexible cable 514 includes electrical wires and tubes to connect the probe 500 to the monitoring unit 13. The cervix probe 500 can be removed relatively quickly from the vagina 16, even while inflated, by drawing the flexible cable 514 proximally. In that regard, the cable should have enough strength and durability to withstand the tension forces during pulling. For this purpose the flexible cable 514 may include a non-expansible fine plastic line (e.g. fishing line) bundled with the electrical wires and tubes to enhance its tensile strength. The flexible cable 514 may have an inter-connector (not shown) and an extension cable (not shown). This arrangement may allow the use of a shorter cable 514. The inter-connector may have an air valve to minimize or prevent air leakage from the balloons when the cable 514 is disconnected from the extension cable. The proximal end of the extension cable 514 has tube/wire connector 92, which plugs into the bedside monitor 13. The proximal portion of the probe 500 may include a docking hole 516 where the distal end of the applicator 530 may engage to hold the probe 500 prior to its application into the vagina 16.

Range-Adjusting Applicators

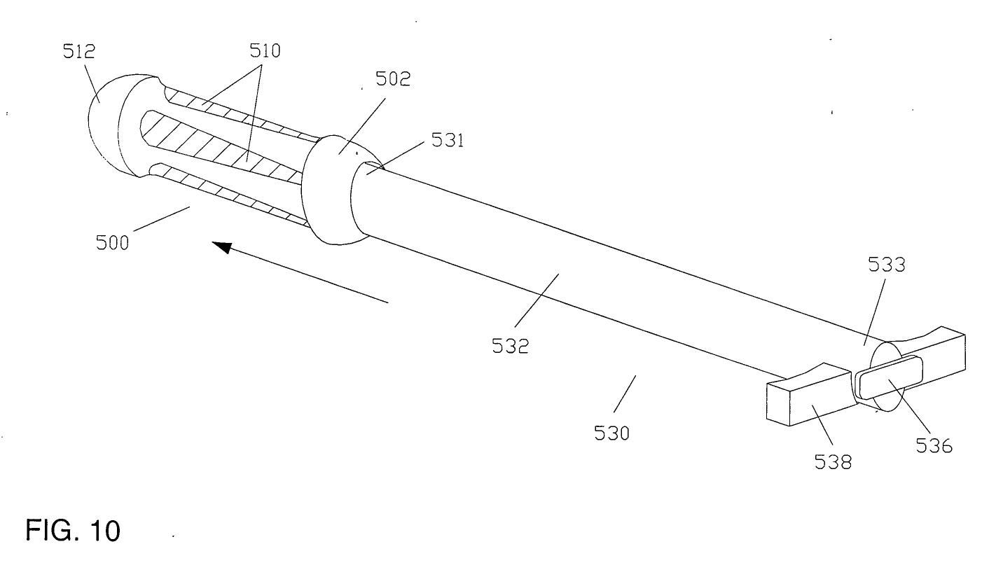

Prior to its application in the patient's vagina, the probe 500 sits on or in the distal end 531 of a rod-shaped applicator 530 as shown in FIG. 10 to facilitate the insertion of the probe 500 into the vagina 16. A proximal end 533 of the applicator shaft 532 has a probe release button 536 to release and deploy the probe 500 from the applicator 530. A distal end 531 of the applicator 530 includes a mechanism to hold the probe 500 until released by the user. The holding mechanism (not shown) may be retractable locking lips that engage into corresponding notches in the probe's docking hole 516, a Velcro attachment pair, a magnetic pair, or by simple friction. Pressing the release button 536 against the wings 538 translates distally a release rod (not shown) within the shaft 532 of the applicator 530 so that the distal tip of the release rod (not shown) pushes the probe 500 forward to disengage from the distal end 531 of the applicator 530 and lodge within the vagina 16. The applicator 530 may also have range-adjusting features to aid the user in placing the probe at a predetermined distance from the cervix 14 as described below. Advantageously, the range-adjusting features maintain the calibration of the camera sensor to physical dimensions in the vagina.

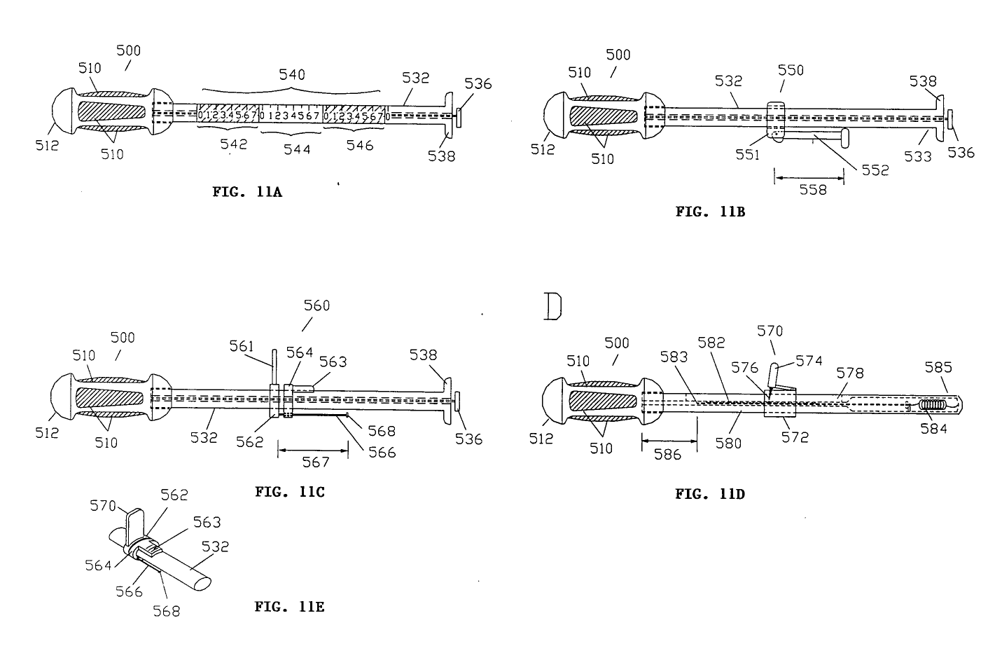

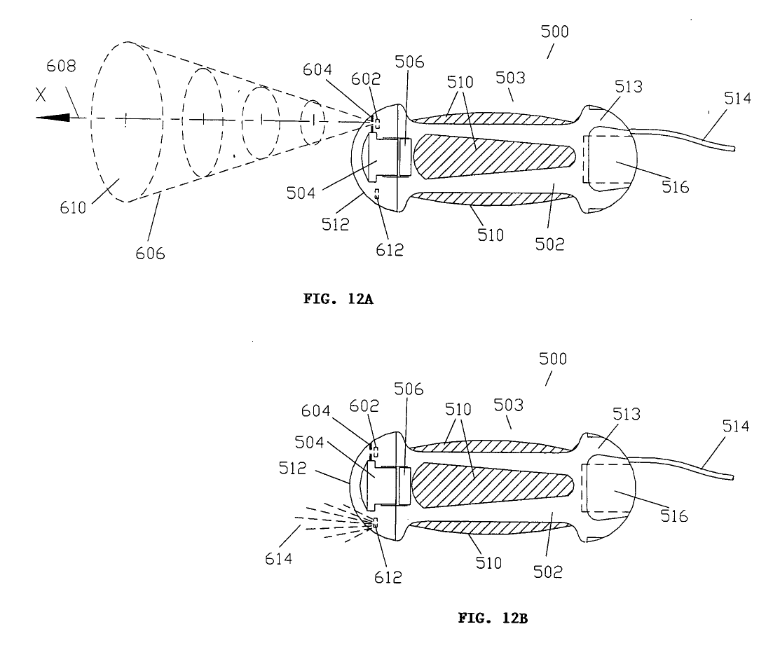

In one embodiment, a range adjuster includes a linear scale 540 printed along a segment of the shaft 532 as shown in FIG. 11a. The linear scale 540 may be numbered, graduated and/or color-coded to assist the operator in positioning the probe 500 at a predetermined distance from the cervix 14. In a typical probe placement procedure, the probe is inserted into the vagina 16 until the distal ends abuts the cervix 14. This may be done under live video guidance from the camera 506 and/or by gently introducing the probe 500 until further distal advancement is blocked by the cervix. The operator then looks at the scale 540 and takes note of the number and color background color closest to the vulva 20. The operator then withdraws the applicator 530 until the next matching number on a different background color barely appears out of the vagina 16. In this manner the probe is placed at the proper distance suitable for deployment and releasable anchor in the vagina 16. For example, the scale shown in FIG. 11a is designed to deliver a positioning distance of a about 14-cm at an accuracy of about 0.5-cm. In the illustrated embodiment, the numbers from 0 to 7 are printed every 0.5-cm on the shaft 532 with their background color changing every 4-cm as shown by the color shades 542, 544 and 546. For example, the scale segment 542 may have a yellow background color, the scale segment 544 may have a sky-blue background color, and the scale segment 546 may have a light pink background color.

Another embodiment of the range adjusting applicator is shown in FIG. 11b, where a range-adjuster 550 includes a ring 551 with a swivel arm 552. Normally, the swivel arm 552 is turned toward the proximal end 533 of the applicator 530 (i.e. proximally away from the probe 500). In a typical probe placement procedure, the probe 500 is inserted into the vagina 16 until reaching the cervix 14. This may be done under live video guidance from the camera 506 or by the inability to further advance the probe distally.

The ring 551 slidably mounted on the shaft is translated along the shaft 532 of the applicator 530 until reaching the vulva 20 (i.e. vaginal opening). The applicator 530 is then slowly drawn proximally from the vagina 16 by the minimum distance necessary to allow the swivel arm 552 of the adjuster to flip distally and rest on the shaft 532. The balloons 510 may then be inflated until a suitable view of the cervix is obtained. Finally, the release button 536 is pressed to release the probe 500 at the vaginal location attained by the foregoing process and the applicator 530 is drawn out of the vagina 16. This procedure enables the probe 500 to be placed at a generally known distance from the cervix 14 as determined by a length 558 of the swivel arm 552 of the range-adjuster 550.

Yet another embodiment of the range adjusting applicator is shown in FIG. 11c, where a range-adjuster 560 includes two rings 562 and 564 tethered to each other by a tensile member or string 566 of a predetermined length. The string 566 secures the rings 562 and 564 to each other such that the rings can separate up to a distance no greater than the length 567 of the string. In that regard, one end may be tied to one ring while the other end passes through an aperture configured in the other ring as shown in FIG. 11c. In a typical probe placement procedure, the probe 500 is inserted into the vagina 16 until it reaches the cervix 14. This may be done under live video guidance from the camera 506 or by determining further distal advancement of the probe is blocked by the cervix. Both rings 562 and 564 are then slid distally along the shaft 532 of the applicator 530 until further distal movement of both rings is blocked by the vulva with tab 561 provided on the more distal ring touching the vulva 20 (i.e. vaginal opening). Then, holding the tab 561 of the adjuster against the vulva 20 with the thumb of one hand, the user grips the shaft 532 of the applicator 530 with the fingers of the other hand while resting the thumb on the tab 563 and drawing the applicator 530 proximally out of the vagina 16 up to the full length 567 of the string 566. The balloons 510 may be then partially or fully inflated until a suitable view of the cervix 14 is obtained. Finally, the release button 536 is pressed to release the probe 500 at the vaginal location and the applicator 530 is pulled out of the vagina 16. This procedure ensures that the probe 500 is placed at an approximately known distance from the cervix 14 as determined by a length 567 of the limiting string 566 of the range-adjuster 560.

Another embodiment of the range adjusting applicator is shown in FIG. 1 id, where a range-adjuster 570 is automatic and requires minimum effort from the user. The range-adjuster has a sliding ring 572 with a pivotable tab 574 controlling a spring-loaded locking pin 576 that rides in a longitudinal groove 578 in a shaft 580 and is adapted to lock against a toothed release-rod 582 generally co-axial with the shaft The release rod 582 is normally retracted into the shaft 580 by a spring 584 such that its distal tip 583 sits inside the shaft 580 by a distance 586 as shown. A distance 586 is equivalent to the desired range at which the probe should be released from the applicator 530.

In a typical probe placement procedure, the probe 500 is inserted into the vagina 16 until reaching the cervix 14. This may be done under live video guidance from the camera 506 or by determining that further distal advancement of the probe is blocked by the cervix. The ring 572 is then proximally slid along the shaft 580 of the applicator toward the vulva 20 until the tab 574 touches the vulva 20. Holding the tab 574 of the adjuster against the vulva 20, the user simply pulls proximally the proximal end 585 of the shaft 580. This action retracts the probe 500 out of the vagina 16 to a predetermined distance 586 where the release-rod 582 starts pushing against the probe 500 to release it from the applicator 530 and into the vagina 16. This procedure generally enables the probe 500 to be placed at an approximately known distance from the cervix 14 as determined by the distance 586 between the distal tip 584 of the toothed release-rod 582 and the distal end of the shaft 580.

Each embodiment of the range adjusting applicators described above assists the user in locating the desired distance from the cervix at which the probe is to be deployed. However, for even greater accuracy, the cervix probe 500 may include a range finder to measure the distance between the probe 500 and the cervix 14. Knowing the magnification of the lens 504, the measured distance may be used to convert the cervix image captured by the camera 506 to real physical dimensions (i.e. cm). The range finder may be of the ultrasonic, laser, or optical type.

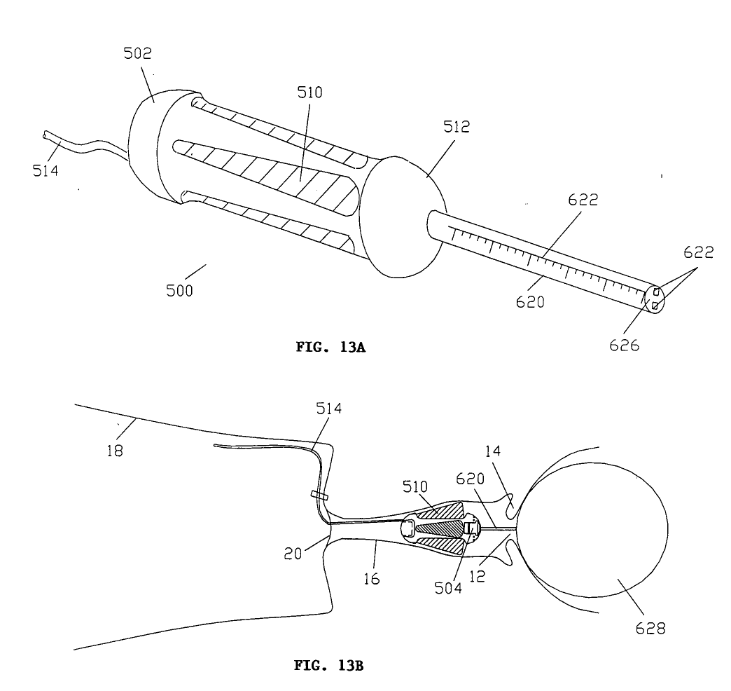

FIG. 12 illustrates one embodiment of the probe 500 having a range finder where a light source 602 and the camera 506 may be used for measuring the range and/or orientation of the probe. The light source 602 is configured to produce high-intensity light that passes thought a hole 604 to emerge as a beam 608 that may have a conical shape 606 with a known spread rate. The projection of the beam 608 on the cervix 14 appears as a bright spot 610 on the cervix that may be imaged by the camera 506. The image of the bright spot 610 may be analyzed by the processor 300 to quantify the area and shape of the bright spot 610 using image-processing algorithms such as segmentation or blob analysis. The size of the bright spot 619 is indicative of the distance between the probe 500 and the cervix 14, while the shape (i.e. circular or elliptical) of the bright spot 619 is indicative of the relative orientation or angle between the probe 500 and the cervix 14. The processor 300 then converts the quantified area and shape of the bright spot 610 to distance and tilt between the probe 500 and the cervix 14.

A second light source 612 emits diffuse light 614 to illuminate the cervix 14 and allow its proper imaging by the probe. The image of the cervix is analyzed as described above using segmentation and blob analysis to identify and measure the opening 12 of the cervix 14. In a typical cervix measurement sequence, the first light source 602 may be illuminated momentarily whereby an image of the bright spot is captured by the camera and analyzed for size and shape to estimate the range and relative orientation of the probe 500 to the cervix 14. After estimating the range, the second light source 612 is illuminated to provide diffuse light 614 for imaging of the cervix 14.

Alternatively, a dual intensity or dual color single light source may be used instead of the two light sources 602 and 612 described above. One intensity level may be used for ranging while the other may be used for imaging.

Another embodiment of the probe 500 shown in FIG. 13a includes a short spacer rod 620 protruding distally from the front shield 512 of the probe 500. A distal tip 626 of the spacer rod 620 includes a reflectance oximetry sensor 622. The spacer rod 620 may include a linear scale 624 or light reflective marks (not shown). The spacer rod 620 is sized so that it does not adversely obstruct the field of view of the wide-angle lens 504 and merely appears as a small spot in the image acquired by the camera 506. The linear scale 624 or light reflective marks are imaged by the camera 506 along with the cervix 14 and image processing may be used to determine an insertion depth of the spacer rod 620 into the cervix opening by counting the visible tics or marks. The tip 626 of the spacer rod 620 is made of a soft rubbery material, preferably silicone, so that it rests relatively softly on a presenting part of a fetus's head 626. The spacer rod 620 may be also configured to buckle or bend under a low compression threshold as an additional safety measure.

In a normal application, the probe 500 is attached to an applicator 530 and inserted under live video guidance from the camera 506 such that the spacer rod 620 is inserted into the opening 12 of the cervix 14. Inflation of the balloon 510 may slightly push the probe 500 toward the cervix 14 such that the tip 626 of the spacer rod 620 gently rests on the presenting portion of the fetus head 628 as shown in FIG. 13b. Provided that the amniotic sac has been ruptured and the mucus plug has already dropped and, the oximetry sensors 622 have good contact with the presenting portion of a fetus head 628, the oximetry sensors 622 can measure oxygenation level of the fetus. The overall conical shape of the probe 500 may gently push the probe 500 toward the cervix 14 thereby enhancing the contact of the oximetry sensors 622 and the presenting portion of the fetus head 628. In addition to carrying the oximetry sensor, the spacer rod 620 may help maintain the probe 500 at an approximately fixed range from the cervix 14 and hence acts as self-ranging feature.

In addition, to the above advantages of the spacer rod 620, the linear scale 624 may be used to measure the length of the cervical canal. Aside from its application during labor, this probe embodiment may be used during routine clinical visits to assess the possibility of preterm labor.

Although the above detailed description describes and illustrates various preferred embodiments, the invention is not so limited. Many modifications and variations will now occur to persons skilled in the art. As such, the preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

1. A system for monitoring dilation of a cervix opening during labor, comprising:
a probe configured to image the cervix opening and provide image data, and
a monitoring unit having a processor and an image display, the processor configured to receive and process the image data to identify the cervix opening and provide data signals representing the cervix opening to the image display for displaying information based on said data signals.

2. A system of claim 1, wherein said data signals include data representing a dimension of the cervix opening.

3. A system of claim 1, wherein the information displayed on the image display includes a graphic image of the cervix opening.

4. A system of claim 1, wherein the information displayed on the image display includes alphanumeric symbols indicating a diameter of the cervix opening.

5. A probe positionable in a vagina for monitoring a cervix during labor, comprising:
a housing adapted for insertion and withdraw from the vagina;
a camera situated in the housing, the camera adapted to capture images of the cervix;
a light source adapted to illuminate the cervix; and
a balloon on the housing, the balloon adapted to inflate around at least a portion of the housing for positioning the probe in the vagina.

6. A probe of claim 5, wherein the housing is configured with a distal end and a proximal end, and the camera is situated a predetermined distance from the distal end of the housing.

7. A probe of claim 6, wherein the probe has a distal end configured to contact the cervix opening when said balloon is inflated.

8. A probe of claim 7, wherein the balloon is configured such that when it is inflated the camera is positioned a predetermined distance from the cervix.

9. A probe of claim 5, further comprising a lens adapted to provide the camera with a wide field of view.

10. A probe of claim 5, further comprising a range detection means for detecting a range between the probe and the cervix.

11. A probe of claim 5, wherein the balloon assumes a generally conical shape when inflated.

12. A probe of claim 5, wherein the balloon assumes a generally spherical shape when inflated.

13. A probe of claim 5, further comprising a handle extending proximally from a proximal end of the housing, the handle configured to facilitate insertion and withdrawal of the probe from the vagina.

14. A probe of claim 12, wherein the handle is a generally flexible structure but adapted to receive a generally rigid member when used to insert the housing into the vagina.

15. A probe of claim 12, wherein the handle is a flexible tube having a lumen extending therethrough.

16. A probe of claim 13, wherein a conduit extends longitudinally along the flexible tube, the conduit configured to pass air into and out of the balloon.

17. A probe of claim 13, wherein the flexible tube is configured with apertures to collect fluids into the tube lumen for drainage out of the vagina.

18. A system for monitoring dilation of an opening of a cervix, comprising:
a probe configured to image the cervix opening and provide image data, and
a processor configured to receive and process the image data to identify the opening of the cervix and provide data on a dimension of the opening.

19. A system of claim 18, wherein the processor is configured to implement blob analysis to identify the opening of the cervix and measure its diameter.

20. A system of claim 18, wherein the processor is configured to implement image segmentation to identify the opening of the cervix and measure its area.

21. A system of claim 20, wherein the processor is configured to convert the area of the cervix opening into a diameter.

22. A system of claim 20, wherein the processor is configured to convert the area of the cervix opening into a diameter by assuming a circular cervix opening model.

23. A system of claim 18, wherein the dimension is a diameter and the processor is configured to implement consecutive diameter measurements to calculate a dilation rate of the cervix opening.

24. A system of claim 18, further comprising an image display that is controlled by the processor, wherein the processor provides to the image display signals for simultaneously displaying images of the cervix at different dilation stages.

25. A system of claim 18, further comprising an image display that is controlled by the processor, wherein the processor provides to the image display signals for simultaneously displaying images of the cervix opening periodically acquired during labor.

26. A system of claim 18, wherein the processor is configured to correct image distortion.

27. A system of claim 26, wherein the image distortion is caused by a lens with uneven magnification between its edges and its center.

28. A system of claim 26, wherein the image distortion is a barrel distortion.

29. A method for monitoring dilation of a cervix opening using a probe, a processor and an image display, comprising:
the probe capturing an image of the cervix opening and providing image data;
the processor receiving and processing the image data to identify the cervix opening, determining a diameter and providing data signals representing the cervix opening to the image display; and
the image display displaying information based on said data signals.

30. A method of claim 29, wherein the processor implements blob analysis to identify the cervix opening and measure a diameter.

31. A method of claim 29, wherein the processor implements image segmentation to identify the cervix opening and measure its area.

32. A method of claim 31, wherein the processor converts the area of the cervix opening into a diameter.

33. A method of claim 31 wherein the processor converts the area of the cervix opening into a diameter by assuming a circular cervix opening model.

34. A method of claim 29, wherein the processor implements consecutive diameter measurements to calculate a dilation rate of the cervix opening.

35. A probe positionable in a vagina for imaging an opening of a cervix during labor, comprising:
a housing configured for insertion into and removal from the vagina;
a camera situated in the housing, the camera adapted to capture images of the opening of the cervix;
a spacer for positioning the probe within the vagina a predetermined distance from the cervix.

36. A monitor of claim 35, wherein the spacer is a flexible rod extending distally from a distal end of the housing.

37. A monitor of claim 35, wherein the spacer is inflatable.

38. A probe for imaging a cervix, comprising:
an imaging member adapted to capture an image of the cervix; and
a cap covering at least a portion of the imaging member, the cap being releasably coupled to the imaging portion for disposal after use with the imaging member.

39. A probe of claim 38, wherein the cap includes a balloon.

40. A probe of claim 38, wherein a portion of the cap is transparent.

41. A probe of claim 40, wherein the cap includes a protective sheath that is adapted for deployment over the imaging member.

42. A system for monitoring dilation of a cervix opening, comprising:
a probe adapted for imaging the cervix opening and generating image data, and
a processor receiving and processing the image data,
wherein the probe has a hydrophobic imaging window.

43. A system for monitoring dilation of a cervix opening, comprising:
a probe adapted for imaging the cervix opening, and generating image data; and
a processor receiving and processing the image data;
wherein the probe has a hydrophobic imaging aperture.

44. A system for monitoring dilation of a cervix opening, comprising:
a probe adapted for imaging the cervix opening and generating image data; and a processor receiving and processing the image data;
wherein the probe has a hydrophobic probe housing.

45. A system for monitoring dilation of a cervix opening, comprising:
a probe adapted for imaging the cervix opening and generating image data; and
a processor receiving and processing the image data;
wherein said probe includes pneumatic means for cleaning its imaging window.

46. A system of claim 45, wherein the processor activates said pneumatic means based on the image data.

47. A probe positionable in a vagina for monitoring dilation of a cervix opening, comprising:
a housing adapted for insertion and removal from the vagina;
a camera adapted for imaging the cervix opening; and
a balloon configured to inflate for widening the vagina to provide the camera with a view of the cervix opening.

48. A probe of claim 47, wherein said balloon is transparent;

49. A probe of claim 47, wherein said balloon generally encapsulates the housing.

50. A probe of claim 47, wherein said balloon is multi-compartmental;

51. A probe of claim 47, wherein expansion of said balloon when inflated is limited by cords;

52. A probe positionable in a vagina for monitoring dilation of a cervix opening, comprising:
a housing adapted for insertion and removal from the vagina;
a camera adapted for imaging the cervix opening; and
a plurality of balloons disposed around said housing;
wherein inflation of said balloons widens the vagina to provide the camera with a view of the cervix opening.

53. A probe of claim 52, wherein said balloons are elongated along a longitudinal axis of the housing.

54. A probe positionable in a vagina for monitoring dilation of a cervix opening, comprising:
a housing adapted for insertion and removal from the vagina;
a camera adapted for imaging the cervix opening; and
a plurality of balloons disposed around said housing;
wherein inflation of said balloons creates channels to allow fluid drainage along a longitudinal axis of said probe.

55. A probe positionable in a vagina for monitoring dilation of a cervix opening, comprising:
a housing adapted for insertion and removal from the vagina;
a camera adapted for imaging the cervix opening; and
a multi-compartmental balloon disposed around said housing;
wherein inflation of said balloon creates channels to allow fluid drainage along a longitudinal axis of said probe.

56. A probe for imaging a cervix, comprising:
a distal imaging member; and
a proximal supporting member;
wherein said distal imaging member and said proximal supporting member are releasably coupled to each other for insertion of the distal imaging member into a vagina by the proximal supporting member and release of the distal imaging member at a suitable position within the vagina.

57. A probe of claim 56, wherein the proximal supporting member includes means for releasing the distal imaging member.

58. A probe of claim 57, wherein the release means includes a longitudinally movable member.

59. A probe of claim 56, wherein the proximal supporting member include means for determining a distance between the distal imaging member and the cervix.

60. A probe for imaging a cervix comprised of:
a distal imaging member;
a proximal elongated member which is generally flexible and configured to receive a generally rigid elongated supporting member for greater rigidity when said probe is inserted into a vagina.

61. A unit for monitoring dilation of a cervix opening, comprising:
a probe adapted for placement in a vagina for imaging a cervix and generating image data, and
a processor configured to receive and process the image data;
wherein the probe includes an oximetry sensor to measure the oxygenation of a fetus through the cervix opening.

62. A unit for monitoring dilation of a cervix opening, comprising:
a probe configured for placement in a vagina for imaging a cervix and generating image data; and
a processor configured to receive and process the image data;
wherein the said probe includes an oximetry sensor that contacts a presenting part of a fetus through the cervix opening to measure oxygenation of said fetus.

63. A unit for imaging a cervix from inside a vagina during labor, comprising:
a probe including:
a first light source that diffusely illuminates the cervix for imaging;
a second light source that illuminates a spot on the cervix for ranging;
a distal imaging member adapted to capture images of the cervix and generate cervix image data representing the cervix and to capture at least one image of the spot and generate spot image data;
a processor configured to receive and process said cervix image data and said spot image data, wherein the spot image data is processed to indicate a distance between said probe and said cervix.
